(19) United States
(12) Patent Application Publication
(10) Pub. No.: US 2016/0303360 A1
(43) Pub. Date: Oct. 20, 2016

(54) Ultrasonic Transducer and Transdermal Delivery System

(71) Applicant: Actuated Medical, Inc., Bellefonte, PA (US)

(72) Inventors: Maureen L. Mulvihill, Bellefonte, PA (US); Kevin A. Snook, State College, PA (US); Rob Van Ess, II, State College, PA (US); Ryan S. Clement, State College, PA (US); Jason A. Showers, Bellefonte, PA (US); Andrew J. Meehan, Warriors Mark, PA (US); Roger B. Bagwell, Bellefonte, PA (US)

(73) Assignee: Actuated Medical, Inc., Bellefonte, PA (US)

(21) Appl. No.: 15/130,665

(22) Filed: Apr. 15, 2016

Related U.S. Application Data
(60) Provisional application No. 62/147,750, filed on Apr. 15, 2015.

(51) Int. Cl. A61M 37/00; H02N 2/00; B06B 1/06; A61N 1/30

(52) U.S. Cl. CPC A61M 37/0092; A61N 1/303; H02N 2/003; B06B 1/0648; A61M 2037/0007

(57) ABSTRACT

A transdermal delivery system including a first passive portion containing a substance for delivery in a reservoir and a second active portion that includes an ultrasonic source. The ultrasonic source includes piezoelectric element(s) that receive electrical stimulation and move radially. A support member fixed to one side of the piezoelectric element(s) restricts movement at that side, so the opposite side expands and contracts. A fulcrum attached to the support member opposite of the piezoelectric element(s) provides an anchor point about which the piezoelectric element(s) bends and flexes upon electrical stimulation. This provides a low ultrasound frequency with a smaller sized transducer than previously known. The active portion is applied to provide electrical stimulation for a certain length of time and removed, whereas the passive portion may remain for a longer duration. A feedback loop also monitors and adjusts the electrical stimulation to maintain a uniform or constant resonance frequency.

### CLAIM OF PRIORITY

The present application claims priority to U.S. Provisional Application having Ser. No. 62/147,750 filed on Apr. 15, 2015, the contents of which are incorporated by reference in its entirety.

### STATEMENT REGARDING FEDERALLY SPONSORED RESEARCH OR DEVELOPMENT

This invention was made with government support under AI080335 awarded by the National Institutes of Health. The government has certain rights in the invention.

### FIELD OF THE INVENTION

The present invention pertains generally to the field of medical devices and more specifically to a system for transdermal delivery of substances (such as but not limited to medication, nutrition and hydration) using ultrasound. At least one embodiment also includes iontophoresis to further enhance transdermal delivery.

### BACKGROUND OF THE INVENTION

Transdermal delivery of substances has been used extensively for low molecular weight molecules where passive diffusion through the skin, such as from a patch affixed to the skin, and into the bloodstream via capillaries beneath the skin surface is possible due to small molecular size. This direct route of transport to the bloodstream provides transdermal drug delivery with multiple advantages over other methods of drug delivery. For instance, the conventional oral route often requires taking large amounts of medication to ensure systemic delivery and sufficient concentrations after first pass metabolism in the gastrointestinal (GI) tract. These large oral doses can result in significant side-effects and complications. Needle injections (e.g., intravenous, intramuscular, or subcutaneous) provide an efficient delivery method, specifically for less stable medications that cannot survive the GI tract or first pass metabolism. However, injection pain, injection site reactions and infections can lead to noncompliance by patients treating chronic illnesses.

However, a significant impediment to adoption of topically applied drugs has been the limitation of permeation through the outermost layer of the skin, the stratum corneum, which is composed of dead, keratinized cells, arranged in a tight, multi-layered “brick and mortar” structure with lipid regions between the layers. Permeation of compounds is related to molecular weight of the compound, with a limit for passive diffusion of approximately 500 Daltons. Despite this passive limit, much research has studied methods for active delivery of large molecule (larger than 500 Daltons) molecules due to the significant potential for reduced side effects, pain and infection risk, as well as the possibility of sustained dosing versus bolus dosing.

One prime candidate for transdermal drug treatment is Human Immunodeficiency Virus (HIV). HIV is a devastating disease that affects thirty-three million people worldwide. The main treatment approach for HIV patients is Highly Active Antiretroviral Therapy (HAART), which uses multiple medications to arrest viral replication. However, the HIV virus can become resistant to the HAART treatment, and salvage drugs must be tried, one of which is enfuvirtide (T-20). T-20 is subcutaneously injected twice daily due to the GI instability and clearance rate of the peptide. While T-20 is highly effective, a major side-effect is an injection site reaction which is experienced by up to 98% of patients. Despite that medication regimens for the management of HIV must be taken with near perfect compliance to maximize benefits and prevent the development of viral resistance, the injection site reaction is painful enough to reduce patient compliance and dissuade many patients from further use.

Peripheral neuropathy (PN) is another example of a condition that can benefit from transdermal medication treatment. PN is a range of disorders resulting from damage to the peripheral somatosensory nervous system, and manifests as debilitating symptoms such as numbness, tingling, abnormal pain sensations and increased sensitivity to normally non-painful or mildly painful stimuli. Even the most effective treatment, oral drugs, is unable to provide clinically meaningful relief for 40-60% of patients, and these often cause side effects due to the high systemic levels required to achieve effect. Topical analgesics such as lidocaine require daily treatments and have mixed success. Recent clinical studies using localized subcutaneous injection of botulinum toxin-A (BTX) demonstrated long-lasting (16+ weeks) and effective pain relief, but subjects required anesthetics before the procedure to attempt to alleviate injection pain. Larger areas of PN, such as that covering the forearm, required at least 40 injections per treatment. Because the size of BTX is so large (150 kDa), a topical transdermal treatment by passive means is not feasible.

A third area in which transdermal treatment could provide benefits is for the effective treatment of skin sores, burns, bedsores, and open wounds. For example, many open wounds do not respond to present treatment practices and never properly heal. In many instances, the circulatory system adjacent to a wound is compromised, thus preventing oxygen from reaching the affected tissues. This lack of oxygen, or prolonged period of oxygen deprivation, is commonly known as hypoxia and can slow or completely stop the natural healing process. The result is permanent, irreversible damage to tissues within and adjacent to a wound, which sometimes leads to the loss of a limb, horrific scarring or disfigurement, and/or death.

A number of enhancement methods have been developed to increase permeability of the stratum corneum, including chemical, electrical (e.g., iontophoresis and electroporation), microdermabrasion, and ultrasound (e.g., sonophoresis or phonophoresis). The most common physical methods to enhance the permeability and delivery of substances across the skin are iontophoresis and ultrasound, for which systems have achieved regulatory approval. Iontophoresis utilizes sustained or oscillatory voltage to actively drive charged molecular agents across the skin, primarily down shunt pathways (e.g., alongside hair follicles). Ultrasound has multiple modes of achieving permeability and delivery enhancement, including the creation of micro-channels as a result of cavitation effects, disruption of lipid layers, convection, acoustic streaming, and other secondary effects such as tissue warming. Simultaneous use of multiple modes of skin permeability enhancement and drug delivery can also have a non-linear amplifying effect on delivery efficiency.

Transdermal delivery devices and methods employing an ultrasound transducer for drug and medication therapies are known within the art. Generally, an ultrasound transducer transforms an electrical signal into an acoustic vibration which, when in communication with the skin, can couple to the skin and temporarily disrupt lipid membranes at the stratum corneum, causing the skin to become more permeable and increasing or enabling delivery of substances into the adjacent tissues and blood system. Prior transdermal delivery devices generally suffered from one or more limitations in design or practice, primarily in the areas of frequency of operation, emitted acoustic intensity and heating (thermal effects).

Typical transdermal ultrasound systems operate above 1 MHz. This is in part because the high stiffness of the piezoelectric ceramics necessitates a thick structure to achieve a low resonance frequency. For example, a 100 kHz thickness-mode piezoelectric plate would need to be more than 2 cm thick with a drive voltage of several thousand volts. A low voltage, portable system would be impractical using this approach. However, Mitragotri and Kost (Advanced Drug Delivery Reviews, 2004) identified multiple studies, including those by Mitragotri (Pharm. Res., 1996), that demonstrate low frequency ultrasound (<100 kHz) enhances transdermal transport many times, up to 1000-fold, relative to high frequency ultrasound.

Furthermore, conventional ultrasonic-based transdermal delivery systems typically use acoustic intensities that are known to damage tissues within the delivery zone, thus resulting in the loss of hair follicles, destruction of sebaceous glands, and necrosis of cutaneous musculature. This is believed to be due to effects from transient cavitation. Cavitation includes the rapid expansion and contraction of gaseous bubbles in response to an oscillating pressure field and broadly includes stable and transient modes. Stable cavitation occurs when a cavity oscillates about its equilibrium radius in response to relatively low acoustic pressures. Transient cavitation occurs when the equilibrium bubble radius greatly varies within a few acoustic cycles causing them to rapidly and violently collapse because of high acoustic pressures. The violent hydrodynamic forces associated with a collapsing bubble can cause highly localized heating of hundreds of degrees, severely damaging biological tissues and releasing free radicals. The cavitation threshold is frequency dependent; the thresholds for stable and transient cavitation are proportional with ultrasound frequency, requiring a lower threshold for lower frequency and necessitating a lower output power to ensure that only stable cavitation, and not transient cavitation, is produced.

Tissue damage can occur not only from high acoustic intensities, but also from thermal heating of the patch. Primary factors contributing to thermal heating in transdermal ultrasound patches are duty cycle of the acoustic waveform, type of waveform, and whether the acoustic source is operating on or off resonance. When operating off resonance, more input power is required to produce the same output acoustic power as when on resonance. Therefore, as a device is driven at its nominal resonance frequency and heating occurs, there may be a shift from that nominal resonance frequency, and, if the drive source is a static frequency, it can result in either a drop in performance, or a further heating effect if the same output acoustic power is maintained. This is because the transducer displacement, and hence acoustic output, decreases significantly off resonance.

Additionally, transdermal devices are capable of extracting interstitial fluid via the interaction between low-frequency ultrasound and tissue adjacent to the epidermis. Applications include glucose monitoring and insulin delivery via devices including a sonicator.

Based on the numerous medical needs and applications, as well as technical issues and limitations with current transdermal delivery technologies and systems, a portable ultrasound-based system is needed. Additionally, development of a low frequency and low power transdermal system that does not produce transient cavitation and has the capability to maintain acoustic output efficiency and consistency through resonance tracking would be a significant advance in the art.

### SUMMARY OF THE INVENTION

The present invention is directed to a piezoelectric ultrasonic source of a new configuration and a transdermal delivery system that uses the same. Specifically, the ultrasonic source includes a piezoelectric element, which may be a flextensional cymbal transducer that is attached on one side to a support member, which is in turn secured to a fulcrum. The support member restricts movement of the piezoelectric element at the point of attachment. When the piezoelectric element is activated, such as by electrical stimulation in the form of voltage or current, the unsecured surface of the piezoelectric element moves in a radial direction relative to the surface secured to the support member. This movement causes a portion of the piezoelectric element to bend or flex relative to the point of fulcrum attachment. The fulcrum therefore acts as an anchor about which the bending or flexing of distal portions of the piezoelectric element occurs. In the bent or flexed position, the piezoelectric element further amplifies the ultrasonic wave emanating and/or reflecting therefrom, thereby permitting low frequency ultrasound (20-50 kHz) to be achieved with a smaller sized piezoelectric element. A cymbal cap may further be secured to the piezoelectric element to provide additional amplification of displacement and a modification to the resonance frequency of the flextensional transducer structure. The configuration of the ultrasonic source described herein provides additional flexibility in the number or orientation of flextensional transducers that fit within the device of a fixed footprint. This provides capability to tailor the profile of the ultrasound energy output by the device, resulting in modifications to output power or treatment area (area of skin permeabilization) underneath the reusable active device.

The present invention is also directed to a transdermal delivery system having two parts—a first or passive portion that includes a substance(s) to be delivered transdermally, and a second or active portion that includes an ultrasonic source as described above. Accordingly, a smaller footprint for a portable transdermal delivery device is achieved. The passive and active portions are selectively attachable and detachable from one another, and may each be provided in a patch form. Further, the passive portion may be disposable for one-time use in delivering the substance(s) contained therein to a particular target tissue, or may be refillable. The active portion, however, is reusable with any number of drug-containing portions. The electronic controller actuates the reusable active device using a preferred voltage signal of a sinusoidal form, though other waveforms such as a square wave, or triangle wave could be used. A sinusoidal signal will be used for exemplary purposes herein, with the understanding that other waveforms could be substituted. The transdermal delivery system of the present invention may further include a control unit in electrical communication with the second active portion, which provides electrical stimulation to activate the piezoelectric element(s), but also may include electrical structures for detecting impedence of the active portion and monitoring the voltage or current emitted and returning after completing the circuit in the active portion, and adjusting the outgoing voltage or current to correct for any change or loss of voltage or current due to impedence of the system. In this manner, the transdermal delivery system includes the capability for maintaining a uniform acoustic intensity output of the ultrasound producing member over a long time duration even with temporal changes in local temperature (such as from self-heating, room-to-body temperature adjustment, or changing environment) by tracking the resonance frequency of the sound producing member, as determined by the frequency of zero phase difference between the output voltage and current of the electronic controller, and dynamically matching the frequency of the output sinusoidal voltage signal to the resonance frequency. This is considered part of the closed loop drive electronics of the transdermal delivery system.

Because the system is portable, treatment may be performed at any location as long as safety allows. The user may activate the active patch through the user interface on the portable electronic controller, which may then run for a time duration specified by the user, and which was previously determined from consultation with a healthcare provider. After the time duration completes and the active patch is inactivated, the user may remove the active patch.

The passive patch function is to contain the compound and be the vehicle that delivers the compound over an extended time preferably 48 hours, more preferably 24 hours, most preferably 12 hours. Whereas the active patch function permeabilizes the skin for a shorter period of time that is then removed at the shorter time point, preferably 50 percent less time than the passive patch usage time, more preferably 75 percent less time than the passive patch usage time, most preferably 92 percent less time than the passive patch usage time. The invention also incorporates electronics with resonance tracking to maintain a consistent acoustic output under different environmental conditions, and to enhance efficiency of permeabilizing the skin and delivering the compound. This system provides the benefits of needle-free compound delivery, electronically controlled compound delivery, system portability and ease of use.

It should be understood that the device and method of the present invention can be used to treat any disease or disorder where a flowable substance is applied to tissue of a subject, such as for chemotherapy treatment, insulin for diabetes, or saline delivery for dehydration, by way of non-limiting examples.

These and other features and advantages of the present invention will become clearer when the drawings and detailed description are taken into consideration.

### BRIEF DESCRIPTION OF THE DRAWINGS

Exemplary embodiments of this invention will be described with reference to the accompanying Figures.

FIG. 1 is a cross-sectional elevation of the transdermal delivery system of one embodiment of the present invention.

FIG. 2a is a perspective view of the active portion of the transdermal delivery system of the present invention.

FIG. 2b is a partial cutaway of FIG. 2a.

FIG. 3a is a cross-sectional elevation of one embodiment of the ultrasonic source of the present invention.

FIG. 3b is a cross-sectional elevation of the ultrasonic source of FIG. 3a depicting maximal concave flexing of the piezoelectric element at resonance.

FIG. 3c is a cross-sectional elevation of the ultrasonic source of FIG. 3a depicting maximal convex flexing of the piezoelectric element at resonance.

FIG. 4a depicts a perspective schematic of the one embodiment of the ultrasonic source that incorporates an array of flextensional stacks with identical piezoelectric polarity directions, and subsequent in-phase deformations, sharing a single fulcrum.

FIG. 4b is a representative graph illustrating the calculated and normalized temporal-peak intensity, and highlighted region within −3 dB of the spatial-peak temporal-peak intensity, produced by the array of FIG. 4a.

FIG. 5a depicts perspective schematic of another embodiment of the ultrasonic source that incorporates an array of flextensional stacks with alternating piezoelectric polarity directions, and subsequent out-of-phase deformations, sharing a single fulcrum.

FIG. 5b is a representative graph illustrating the calculated and normalized temporal-peak intensity, and highlighted region within −3 dB of the spatial-peak temporal-peak intensity, produced by the array of FIG. 5a.

FIG. 6a presents exemplary data of the concentration of T-20 in porcine plasma after the first 90 mg treatment with either transdermal delivery using a combination of said embodiments of the invention or subcutaneous injection.

FIG. 6b presents exemplary data comparing the concentration of T-20 in porcine plasma over a 30 day period when treating with either transdermal delivery using a combination of said embodiments of the invention or conventional subcutaneous injection, as measured before re-dosing.

FIG. 7 apresents exemplary summary data of the transepidermal water loss from porcine skin at the treatment site and a control site prior to the first treatment and on Day 30, after 29 days of twice-daily treatments with saline only using a combination of said embodiments of the invention.

FIG. 7b presents exemplary summary data of the transepidermal water loss from porcine skin at the treatment site and a control site prior to the first treatment and on Day 30, after 29 days of twice-daily treatments with T-20 using a combination of said embodiments of the invention.

FIG. 8a depicts a schematic diagram of the control system for maintaining a uniform acoustic intensity output of the ultrasound producing member.

FIG. 8b depicts a schematic diagram of the scaled sinusoid from a lookup table of the control unit.

FIG. 8c depicts a schematic diagram of the impedence matching of the control unit.

FIG. 9 shows the measured spatial-peak temporal-peak intensity of a reusable active device driven at 15% duty cycle by the control box over 30 minutes as measured by a calibrated hydrophone in water.

FIG. 10 depicts one embodiment of the transdermal delivery system including electrodes for the combined use of ultrasound stimulation and iontophoresis.

FIG. 11 depicts another embodiment of the transdermal delivery system including electrodes for the combined use of ultrasound stimulation and iontophoresis.

FIG. 12 depicts an array of active portions of the transdermal deliveyr system of the present invention electrically connected in an array.

Like reference numerals refer to like parts throughout the Figures.

### DETAILED DESCRIPTION

The present invention is directed to a transdermal delivery system and ultrasonic source for same. As used herein, the terms “vibration” and “oscillation” may be used interchangeably, despite the fact that “vibration” may in some cases specifically relate to mechanical movement, whereas “oscillation” is not limited to mechanical movement. Similarly, “vibration” and “wave” may be used interchangeably herein, despite the fact that “vibration” may pertain to the mechanical movement of an object and “wave” may pertain to a form of energy emanating or resulting from a mechanical vibration. It should be understood that “waves” or “vibrations” can propagate through matter according to known principles of physics. The terms “ultrasound,” “ultrasonic,” “sonic” and “acoustic” may also be used interchangeably, despite the fact that “ultrasound” may be considered to be a subtype of “sound” in a particular frequency range, namely, greater than 20 kHz.

The present invention relates to a compact, reusable active device that produces low-frequency ultrasound, which can be used to permeabilize skin and deliver compounds such as medications. The effectiveness of the invention as described, for example, in the aforementioned preferred embodiments, uses the advantages of both bending modes in piezoelectric materials and flextensional actuators to reduce the size of the invention and provide amplification in displacement, and subsequently acoustic output. The transdermal delivery system as described herein may be used to deliver transdermal compounds in a live being such as a human or an animal, herein the intended user. The present invention may be in some aspects of some exemplary embodiments a control box coupled to a reusable active patch component that is in turn coupled to a one-time use or reusable passive patch component that is coupled to the skin, the combination of which work together to permeabilize the skin and deliver at least one compound such as, but not limited to, medications, nutrition, and/or hydration to the intended user.

Because the system is portable, treatment may be performed at any location as long as safety allows. The treatment may consist of a first application of the passive patch component to the skin of the user, followed by a second application of the active patch to the opposite side of the passive patch. The user may activate the active patch through the user interface on the control box, which may then run for a time duration specified by the user, and which was previously determined with input from a healthcare provider. After the time duration completes and the active patch is inactivated, the user may remove the active patch from the passive patch. The passive patch may remain on the skin of the user for an additional time duration as previously determined with input from a healthcare provider. During this time duration, the user is free to perform other activities.

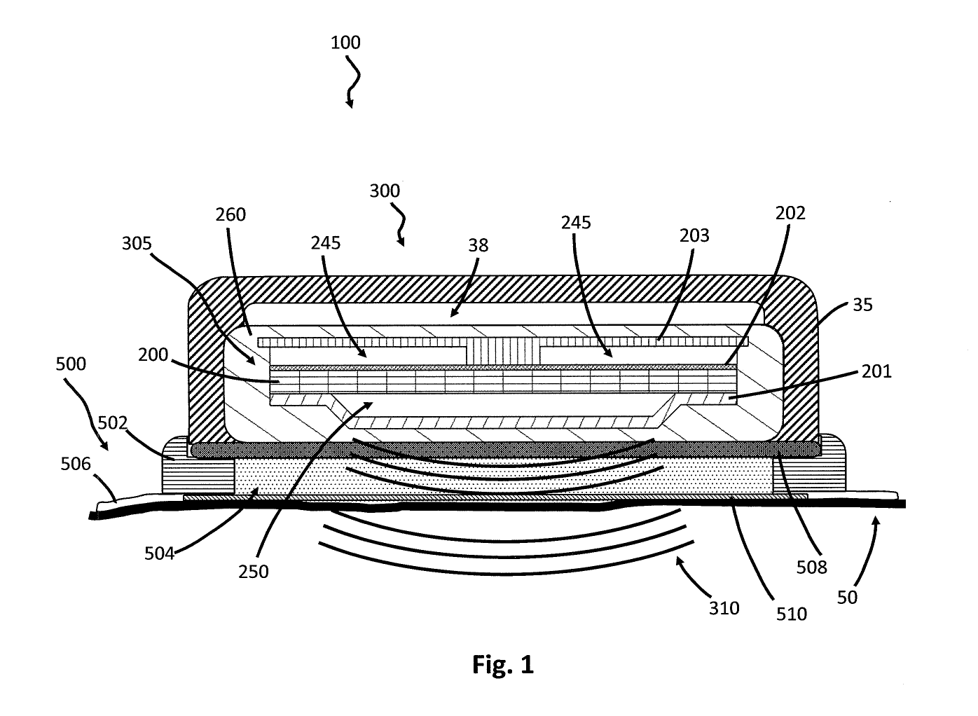

FIG. 1 shows one embodiment of the transdermal delivery system 100 of the present invention. The transdermal delivery system 100 includes a first portion 500, which may also be referred to herein as a passive portion, component or patch. The first passive portion 500 may include at least one reservoir 502 containing at least one substance 504 to be delivered via the device into tissue 50, such as skin.The compound or substance 504 may be any drug, medication, nutrient, hydration, or other type of molecule, and may be in the form of a liquid, gel, or paste, in non-limiting examples. For instance, the substance 504 may include enfuvirtide, gabapentin, botulinum toxin, nutrition, hydration, therapeutics, etc., and may be large (>500 Dalton) or small (<500 Dalton) sized molecules. The reservoir(s) 502 are sized to accommodate and retain the substance(s) 504 therein until driven out by ultrasonic waves. The first passive portion 500 may be placed in contact with the tissue 50 which is the target of delivery. In some embodiments, the first passive portion 500 includes a contacting membrane or adhesive 506. This membrane 506 may be a bio-compatible adhesive, or is at least biologically inert. The adhesive may be disposed along the membrane side that couples directly to the skin. This adhesive could be made from, but not be limited to, the families of polyacrylates (acrylates), polyisobutylene (PIB) or polydimethylsiloxane (silicone). The first passive portion 500 may further include an ultrasonic coupling vehicle 508, such as but not limited to ultrasonic gel, in some embodiments. This ultrasonic coupling vehicle 508 may provide a mechanical or sonically transmissible connection between the first passive portion 500 and a second active portion 300 (described in greater detail hereinafter), and more specifically with an ultrasonic source therein, for the faithful transmission of ultrasound waves 310 from the second active portion 300 where they are generated into the first passive portion 500.

In one embodiment, the first passive portion 500 may further include a membrane 510 disposable in contact between the tissue 50 and the reservoir 502. The membrane 510 may provide a two-way flow of the substance(s) 504 into and out of the reservoir 502, but more preferentially provides a one-way flow of the substance(s) 504 out of the reservoir 502. The membrane 510 may be permeable or semi-permeable to the substance(s) 504, such that application of ultrasonic waves or vibrations 310 permeabilizes the membrane 510 to the substance(s) 504, at least for a period of time that may be for the duration of the application of the ultrasonic waves 510 or for a certain amount of time thereafter. In some embodiments, the membrane 510 may be made from a bio-compatible film material that dissolves when in contact with a predetermined stimulus, such as but not limited to temperature or pH of the skin. In still other embodiments, the passive patch 500 component may further include a sensor to determine one or more conditions within the patch or skin contacting the patch indicative of damage or irritation.

The transdermal delivery system 100 further includes a second active portion 300, which may also be a patch. This second active portion 300 is positioned adjacent to, and in at least one embodiment, in contact with, the first passive portion 500 of the transdermal delivery system 100. In at least one embodiment, this placement positions the first passive portion 500 between the tissue 50 and the second active portion 300. The second active portion 300 includes at least one ultrasonic source 305 that generates ultrasonic waves 310. The second active portion 300 is positioned adjacent to and in communication with the first passive portion 500 so that the ultrasound source 305 is in transmitting communication of the ultrasonic waves 310 through the reservoir 502 and substance(s) 504 and into the tissue 50, as shown in FIG. 1.

In at least one some exemplary embodiment, the ultrasonic source 305 may be a flextensional transducer, such as a cymbal transducer. Flextensional transducers are compact and thereby compatible within micro-patch devices. Cymbal-shaped flextensional transducers, like those described by Newnham et al. in U.S. Pat. No. 5,729,077 entitled Metal-Electroactive Ceramic Composite Transducer, use metal end-caps to enhance the mechanical response of a piezoceramic disk to an electrical input. In a typical cymbal transducer, high frequency radial motion within a disk composed of a piezoelectric ceramic is transformed into low frequency (20-50 kHz) displacement motion through a cap-covered cavity. A cymbal transducer takes advantage of the combined expansion in the piezoelectric charge coefficient d₃₃, representing induced strain in direction 3 per unit field applied in direction 3, and contraction in the d₃₁, representing induced strain in direction 1 per unit field applied in direction 3, by a piezoelectric ceramic, along with the flextensional displacement of the metal end-caps. The end-caps about the ceramic disk enable both longitudinal and transverse responses to contribute to the strain in the desired direction, creating an effective piezoelectric charge constant (d_{eff}) according to the equation

d_{eff}=d₃₃+(−A*d₃₁)

where A is the amplification factor of the transducer which can be as high as 100.

In at least one embodiment, the second active portion 300 may include a housing 35, such as a single encapsulated body. As shown in FIG. 1, the encapsulated body or housing 35 may have a hard cover over the top side (the side that is farthest from the skin) to provide both mechanical impact resistance and provide an air gap 38 on the top side of the encapsulated body for maximal reflection of acoustic waves towards the bottom side (the side that is closest to the skin). The housing 35 may be of any suitably rigid material, such as metal, but is more preferentially plastic such as polyoxymethylene, polyvinyl chloride (PVC) or polypropylene. In some embodiments, the housing 35 may define a patch, such that the transdermal delivery system 100 is a dual-patch system where one patch is active and includes the ultrasound source 305 and the other patch is passive and includes the reservoir 502 with drug or other substance 504 for delivery. In at least one embodiment, the first passive portion 500 is attached or secured to the skin 50 or other target tissue of a subject first, and the second active portion 300 may be placed over or attached to the first passive portion 500 during activation. When activation is no longer desired or required, such as after 10 min-2 hours for example, the second active portion 300 or patch may be removed from the first passive portion 500, which may remain on the skin 50 or tissue for a longer period of time, such as 24-48 hours in at least one embodiment. Therefore, the second active portion 300 or patch may be selectively releasable from the first passive portion 500 or patch. In a preferred embodiment, the first passive patch 500 is disposable or intended for one-time use. In other embodiments, however, it may be refillable, such as by refilling the reservoir 502 with additional substance 504 when depleted. The second active portion 300 or patch is preferably reusable, and may be used on any number of passive patches 500 over time.

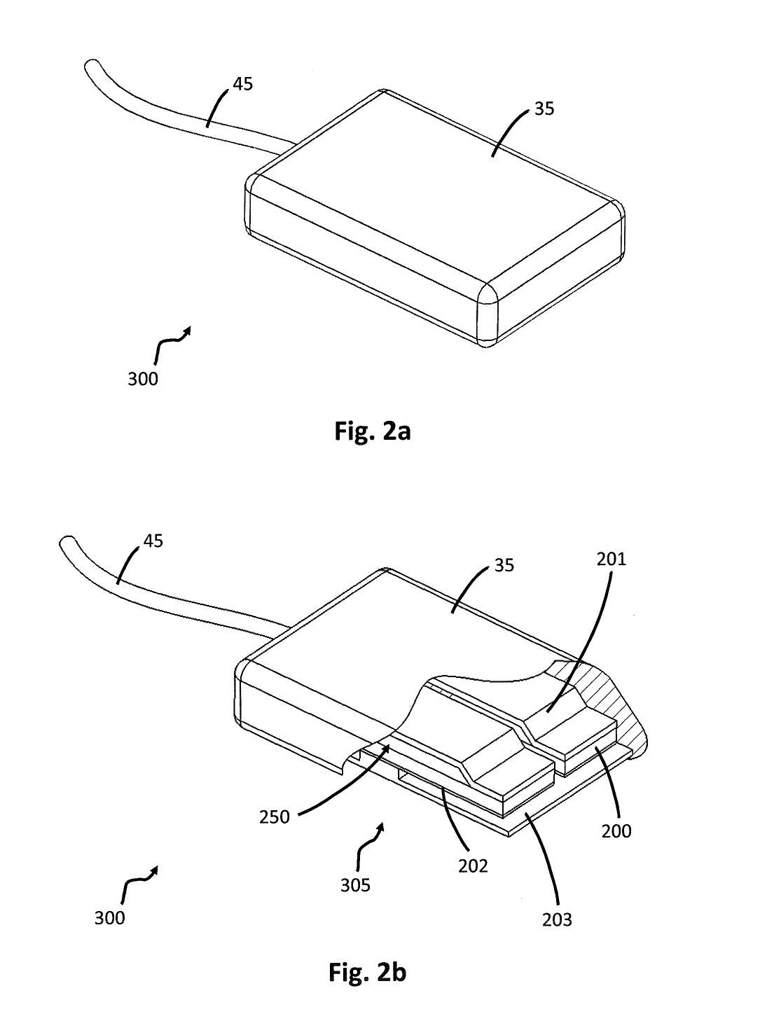

As seen in the embodiments of FIGS. 2a and 2b, the second portion 300 may be encased in a rigid housing 35, and includes a plurality of ultrasound sources 305. The second portion 300 further includes a connection 45 providing electrical communication of the second portion 300 to a control unit (not shown). The control unit provides the electrical stimulation, such as voltage or current, to stimulate the ultrasound source(s) 305 of the second active portion 300. In at least one embodiment, the control unit may further include circuitry, such as microcircuitry, that detects the impedence of the second active portion 300 and calculates or detects the difference between the electrical stimulation supplied to the second active portion 300 and the current or voltage returning from the second active portion 300 upon completion of the electrical current through the second active portion 300. If the difference of the electrical current or voltage returning from the second active portion 300 is any value other than zero, the circuitry may also adjust subsequent electrical stimulation provided to the second active portion 300 to compensate for the difference in electrical current or voltage returning. In this manner, the control unit may monitor and/or maintain a uniform electrical stimulation over the duration of activation of the ultrasound source 305, with the goal being maintaining the ultrasound source 305 on resonance frequency.

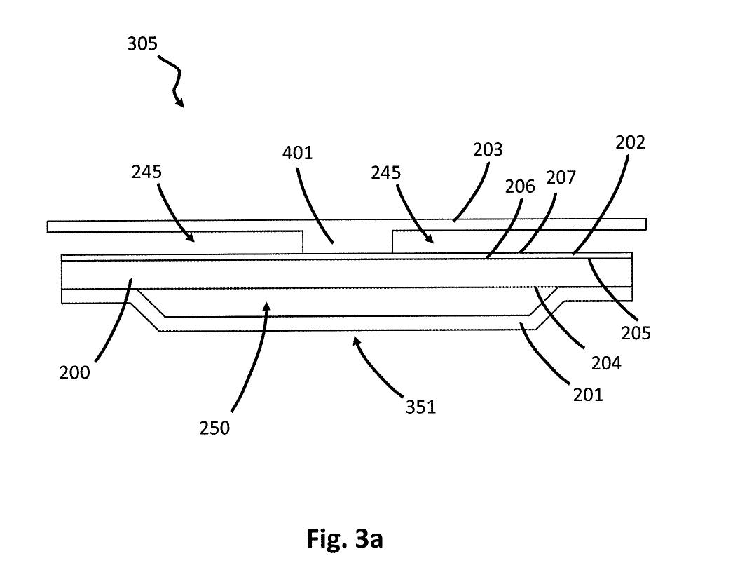

Turning now to FIGS. 1 and 3a, the ultrasound source 305 in the active patch 300 may be a flextensional transducer(s), such as a cymbal transducer. More generally, the ultrasound source 305 includes at least one piezoelectric element 200, such as a piezoelectric ceramic driving cell disposed within a frame, platen, housing, end-caps or other geometry which amplifies the transverse, axial, radial or longitudinal motions or strains of the piezoelectric element 200 in one direction to obtain larger displacement in a second or a preferred direction (such as toward the skin or tissue), than otherwise achievable with the piezoelectric ceramic alone. The piezoelectric element 200 may consist of, but is not be limited to, PZT, PMN-PT, lead titanate, lead zirconium niobate-lead titanate (PZN-PT), or barium titanate. The piezoelectric element 200 has a first surface 204 and an opposite second surface 205.

A support member 202 is secured to the second surface 205 of the piezoelectric element 200 to receive and restrain the second surface 205 as described below with reference to FIGS. 3b and 3c. The support member 202 has a first side 206 that secures to the piezoelectric element 200, and an opposite second side 207, as shown in FIG. 3a. The support member 202 may comprise any suitable material that is sufficiently rigid to support the piezoelectric element 200, and yet also sufficiently flexible to permit vibration of the piezoelectric element 200 upon actuation by electrical stimulation to produce ultrasonic waves 310.

In at least one embodiment, the ultrasonic source 305 may further include a cymbal cap 201 of a type used with cymbal transducers. The cymbal cap 201 creates a cymbal gap 250 between the outer limit of the cymbal cap 201 and the first surface 204 of the piezoelectric element 200. This cymbal gap 250 serves to further amplify the ultrasonic waves or vibrations 310 produced from the piezoelectric element 200 when activated. This enables a higher permeation rate of compounds per patch size by increasing the area of ultrasound treatment underneath the patch. The cymbal cap 201 may be connected to the piezoelectric element 200 at co-terminal ends thereof, such as with end-caps (not shown). The end-cap materials where can include, but are not limited to, brass, aluminum, steel, titanium, and Kovar™, a registered trademark of CRS Holdings, Inc. of Wilmington, Del. Metal end-caps also provide additional mechanical stability, ensuring a longer effective lifetime for the transducer. End-caps could include a variety of profiles and shapes including, but not be limited to, a circular, rectangular, square, hexagonal or triangular shape.

The ultrasonic source 305 further includes at least one fulcrum 401 secured to the second side 207 of the support member 202 and opposite of the piezoelectric element 200, as seen in FIGS. 3a-3c. In at least one embodiment, the fulcrum 401 may include an anchor substrate 203 that may be integrally formed with the fulcrum 401 or may be a separate component thereof. The fulcrum 401 and anchor substrate 203 together may comprise a mass that is more than the mass of the remaining components of the ultrasonic source 305, namely, the piezoelectric element 200, support member 202, cymbal cap 201 and end caps. For instance, in at least one embodiment, the fulcrum 401 and anchor substrate 203 together have a mass that may be more than twice the mass of the remainder of the ultrasonic source 305. Due to this additional mass, the fulcrum 401 restricts the movement of at least a portion of the piezoelectric element 200 when the piezoelectric element 200 is activated.

In at least one exemplary embodiment, the ultrasonic source 305 such as flextensional transducers in the reusable active patch component 300 within a single encapsulated body or housing 35 could use a different vibrational mode or flextensional form factor and use a single cymbal cap 201. By using a unimorph or bimorph transducer instead of a typical piezoelectric plate, a bending mode may be introduced to the ultrasonic source 305 that reduces the effective frequency constant of the transducer, resulting in a shorter piezoelectric 200 that achieves the same resonance frequency as a longer one operating in the pure transverse length-extensional mode.

For example, the bending mode is illustrated in FIGS. 3b and 3c. Here, a unimorph flextensional transducer or ultrasonic source 305 is employed. When electrical stimulation is applied to the ultrasonic source 305, and more specifically to the piezoelectric element 200, the piezoelectric element 200 will begin to move radially. The second surface 205 of the piezoelectric element 200 is attached or secured to the first side 206 of the support member 202. This attachment restricts the movement of the second surface 205 of the piezoelectric element 200, such that only the first surface 204 of the piezoelectric element 200 is free to move upon electrical stimulation or activation. When an electrical stimulation is received by the piezoelectric element 200, such as electrical voltage or current, the unrestricted first surface 204 moves radially relative to the restricted second surface 205. As used herein, “radially” means along a plane substantially parallel to the support member 202, such that the first surface 204 of the piezoelectric element 200 stretches or contracts according to the polarity of the first surface 204 and the electrical charge being applied. For instance, a positive charge applied to a first surface 204 having a positive polarity may cause the first surface 204 to expand, and a negative charge applied to the same first surface 204 would contract, and vice versa. Of course, this is provided for illustrative purposes only. It is within the spirit and scope of the invention that in another embodiment, a positive charge applied to a first surface 204 having a positive polarity may cause the first surface 204 to contract, and a negative electrical charge could cause it to expand, and vice versa.

When the first surface 204 of the piezoelectric element 200 contracts upon electrical stimulation or activation, as depicted in FIG. 3b, the piezoelectric element 200 flexes. Specifically, the movement of the first surface 204 relative to the fixed second surface 205 may be a contracting or shrinking motion, such that the first surface 204 is shorter than the second fixed surface 205. In combination with the fulcrum 401, this movement causes at least one portion of the piezoelectric element 200 to move longitudinally relative to the fulcrum 401, as indicated by the arrows in FIG. 3b in one example. In at least one embodiment, the fulcrum 401 is located centrally along the piezoelectric element 200, such that the distal ends of the peizoelectric element 200 bend or flex about the fulcrum 401. As used herein, “distal” means further from the fulcrum 401. The fulcrum 401 therefore acts as an anchor for the peizoelectric element 200, about which the piezoelectric element 200 bends and flexes upon activation. Because the fulcrum 401 and anchor substrate 203 collectively have more mass than the remainder of the ultrasonic source 300, the piezoelectric element 200 moves and vibrates relative to the fulcrum 401 about the point of attachment. The cymbal cap 201 may also deflect or flex accordingly as it may be joined at the ends to the peizoelectric element 200. In the case of flexion, the cymbal cap 201 may bow out, thereby increasing the cymbal gap 250. This therefore affects the ultrasonic waves 310 produced by the piezoelectric element 200.

Alternatively, as shown in FIG. 3c, when the first surface 204 of the piezoelectric element 200 expands upon electrical stimulation or activation, the piezoelectric element 200 bends. Here, the outer or distal ends of the piezoeletric element 200 bend according to the arrows shown in FIG. 3c relative to the fulcrum 401. The cymbal cap 201 may consequently contract, decreasing the cymbal gap 250 and affecting the ultrasonic waves 310 produced.

Referring to FIG. 1, the ultrasonic source 305 may be enclosed in a housing 35 of the active second portion 300 of the transdermal delivery system 100. Each of the components of the ultrasonic source 305 may be encapsulated or surrounded by potting material, such as is used in flextensional transducers. This potting material allows the piezoelectric element 200 to vibrate therein upon electrical stimulation, and further permits the bending and flexing motion of the piezoelectric element 200 around the fulcrum 401.

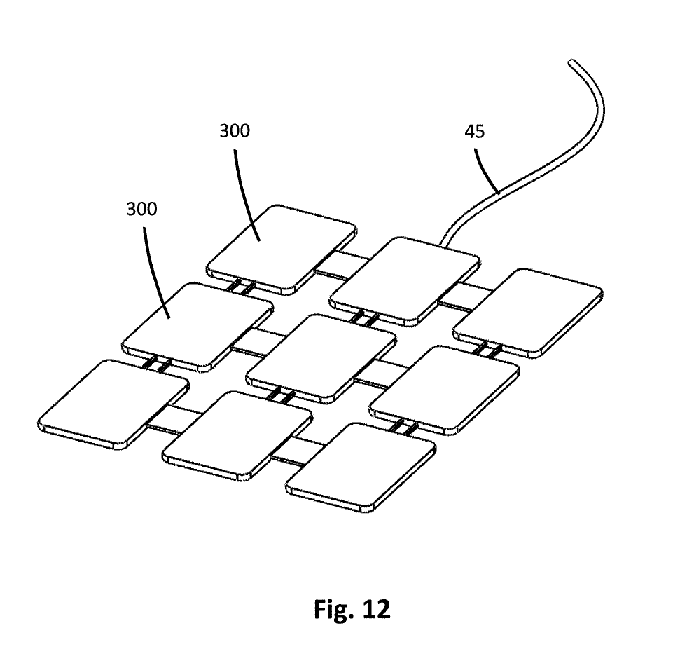

In at least one embodiment, the reusable active patch component 300 includes a plurality of ultrasonic sources 305 within a single encapsulated body or housing 35. Each ultrasonic source 305 includes at least one piezoelectric element 200, and each piezoelectric element 200 includes one portion (such as one surface) having positive polarity and another portion (such as an opposite surface) having negative polarity. The ultrasonic source 305 may be assembled such that the first surface 204 of the piezoelectric element 200 is the positive pole or the negative pole, depending on the desired bending motion for the electrical stimulation provided. Moreover, a second active portion 300 may include a plurality of piezoelectric elements 200 arranged adjacent to one another. Adjacent piezoelectric elements 200 may be electrically connected in an array, such as shown in FIG. 12, and may be connected in series or parallel as is understood in the electrical arts.

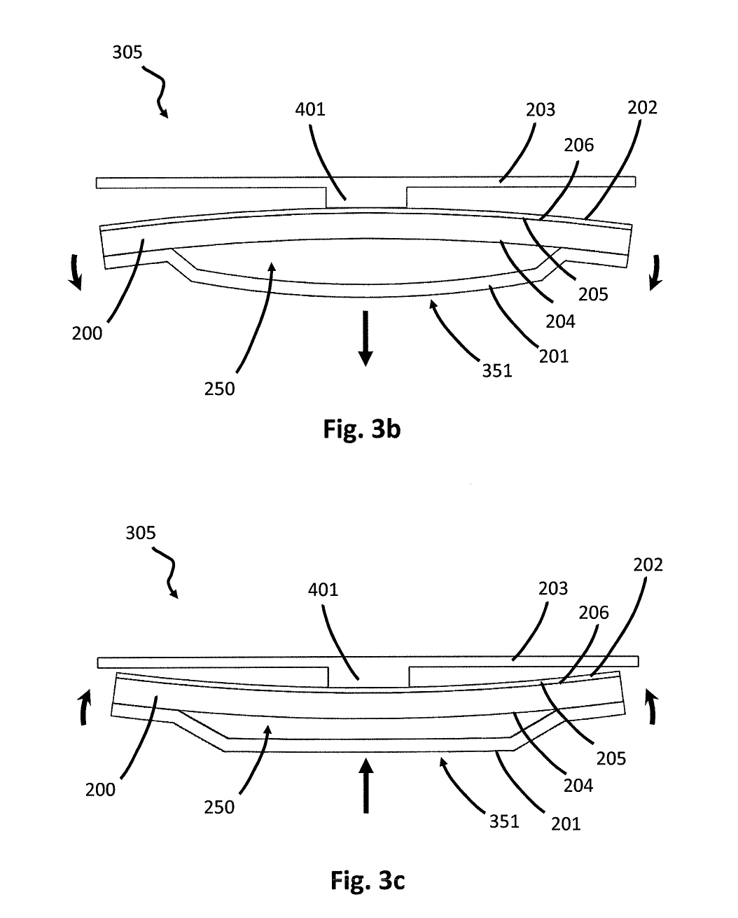

Since each piezoelectric element 200 has its own independent polarity, adjacent piezoelectric elements 200 may be arranged in-phase with each other, as shown in FIG. 4a, so that adjacent piezoelectric elements 200 have similar polarity arrangements. FIG. 4b shows an acoustic radiation field resulting from such an arrangement where all piezoelectric elements 200 are in phase. A spatial impulse response simulation was run using 5 array elements of 22 mm by 3 mm with a 4 mm pitch and an input sinusoidal 5-cycle tone burst of 26 kHz.

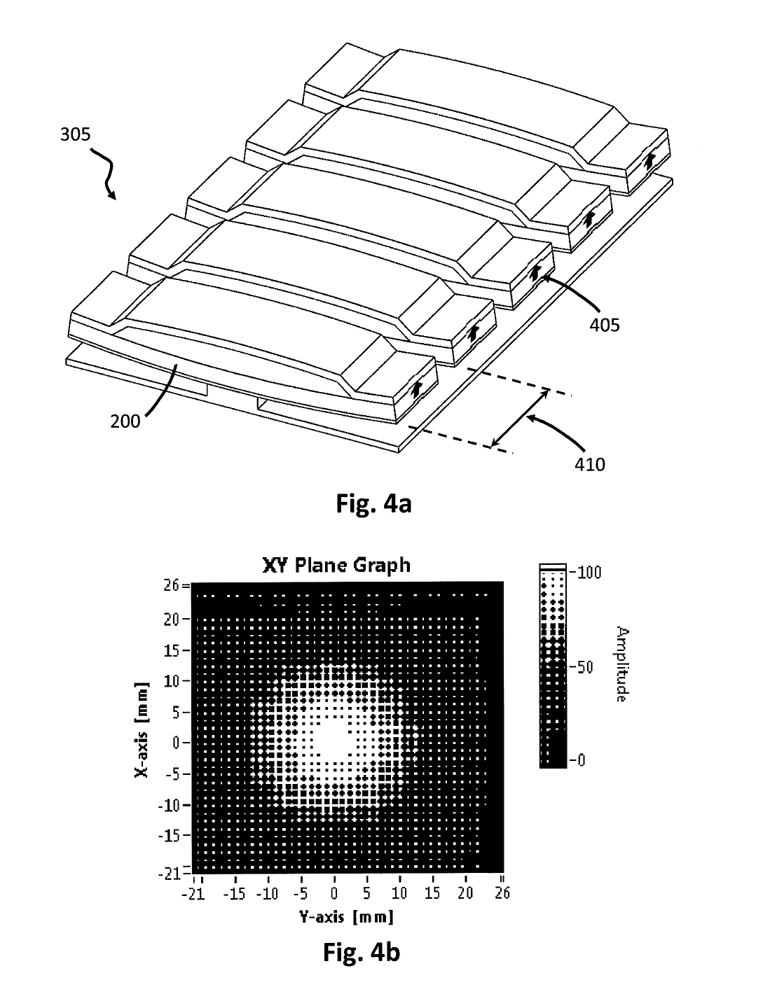

In at least one other embodiment, as shown in FIG. 5a, the ultrasonic source 305 may include a plurality of piezoelectric elements 200 in alternating phase between adjacent transducers such that the displacements of adjacent ultrasonic sources are out of phase by 180 degrees. Using a pitch, or dimensional spacing between element centers in the width direction, of approximately one-quarter wavelength, the constructive and destructive interference of the elements in the array combine such that multiple intensity peaks form at set angles from the normal direction like a diffraction grating. A pitch can be chosen so that the acoustic intensity peaks overlap and create an overall larger region of relatively uniform acoustic intensity at the skin surface relative to using in-phase adjacent ultrasonic sources. This also enables a higher permeation rate of compounds per patch size by increasing the area of ultrasound treatment underneath the patch. This may be achieved by maintaining the same pieozelectric polarity direction between piezoelectric elements 200 or flextensional transducers and alternately wiring positive or negative leads together, or more preferentially placing adjacent piezoelectric elements 200 with alternating piezoelectric polarity directions and wiring a single positive lead and single negative lead. This acts similarly to a diffraction grating device, which produces a response at specific off-angles, when elements of an array are of a specific spacing and/or electrical phasing. By using a pitch spacing of less than one-quarter wavelength (<λ/4), as seen in FIG. 5a, and by switching polarity on adjacent piezoelectric elements 200, at a distance of 3 mm, a 20% increase in acoustic area was achieved as defined by a region with intensity within −3 dB of the spatial peak temporal peak intensity, as shown in FIG. 5b. The end result is a wider range of effect of the ultrasonic waves 310, and therefore, more effective transdermal delivery over a wider range of area despite the same size transdermal device 100.

As noted previously, piezoelectric elements 200 may be connected electrically in parallel and physically aligned in a single plane, resulting in the direction of all transducers being parallel, or may have a slight physical rotation such that the direction of all transducers are not parallel to modify the acoustic intensity profile. Accordingly, the active patch component 300 may have integrated interconnections to allow additional active patch components 300 to be placed in electrical parallel, and increase the overall area of treatment (skin permeability enhancement and compound delivery), as seen in FIG. 12. The electrical connections would provide positive and negative connections for electrical stimulation, as previously discussed.

The practical effect of the transdermal delivery system 100 of the present invention can be further understood by reference to the following non-limiting example:

### EXAMPLE

### In Vivo Administration of T-20

A 30-day, in vivo porcine study with twice-daily treatments to mimic clinical use was performed to study the bioavailability of T-20 delivered via an embodiment of the invention as compared to conventional subcutaneous injection. The study also was used to evaluate the longer-term effects of transdermal ultrasound application to the skin and whether changes in the structure or performance could be seen. The passive skin permeability to T-20 (molecular weight: 4,492 Daltons) is essentially zero (below the detection limit).

Materials and methods: In vivo experiments were performed using domestic pigs of approximately 95-115 pounds starting weight. Pigs were kept in runs and allowed free movement. They had full access to water and regularly scheduled feedings. Animals were not anesthetized and were allowed free movement in runs or cages during treatments. Before administration, Fuzeon® (Genentech) vials were reconstituted to a concentration of 90 mg T-20 to 1 mL water for subcutaneous injections and 90 mg T-20 to 1.5 mL water for transdermal delivery. A wound dressing (Mepore, M\ddot{0}lnlycke USA) with a hole cut-out and surrounding silicone reservoir was adhered to the animals for transdermal delivery, allowing the reconstituted solution in the reservoir to directly contact the skin. The ultrasound device was placed over the reservoir, and ultrasound (approximately 25 kHz frequency, 90 mW/cm², 150 msec pulses every second) was applied for 30 minutes. Subcutaneous injections were given via 20 gauge needle. Two general areas on the animals' backs were used for treatments; one area each for morning and evening.

Blood samples were taken at multiple times the first day of treatments and at regular intervals thereafter. Analysis for plasma concentration of T-20 was performed using mass spectrometry (University of Alabama Birmingham). Transepidermal water loss (TEWL) was measured (Tewameter® TM300, CK Electronics) at the site of treatment and a control site on each animal, and provided a measure of skin function. Histology samples were taken post-study.

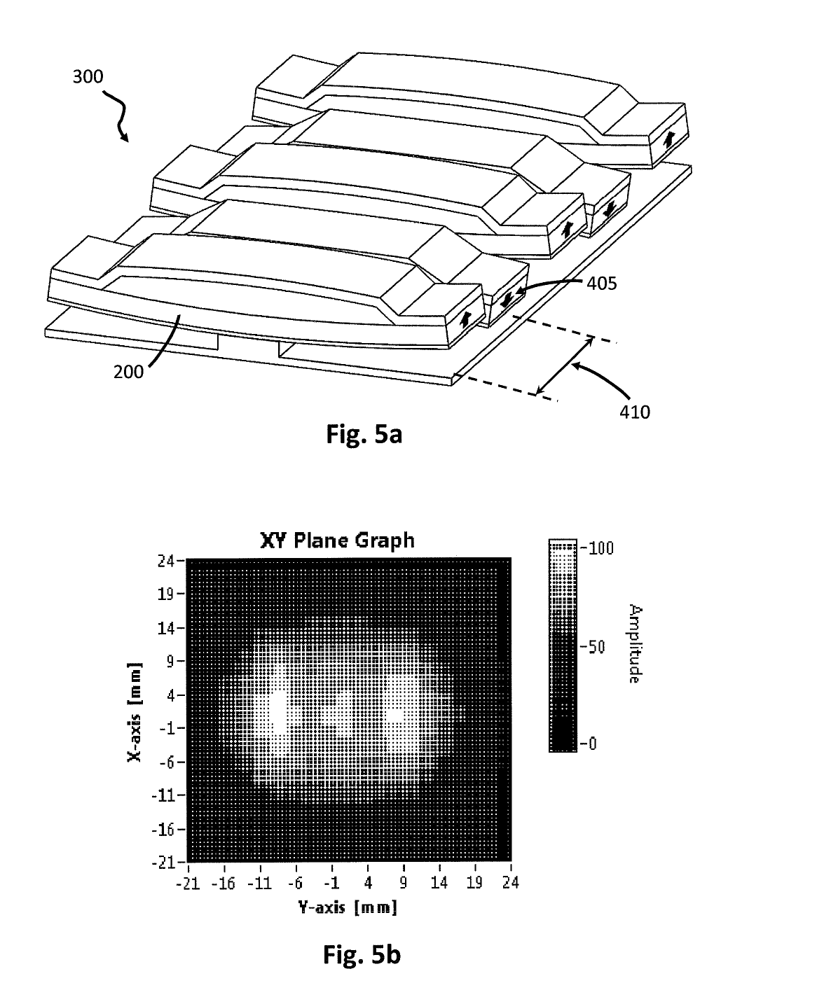

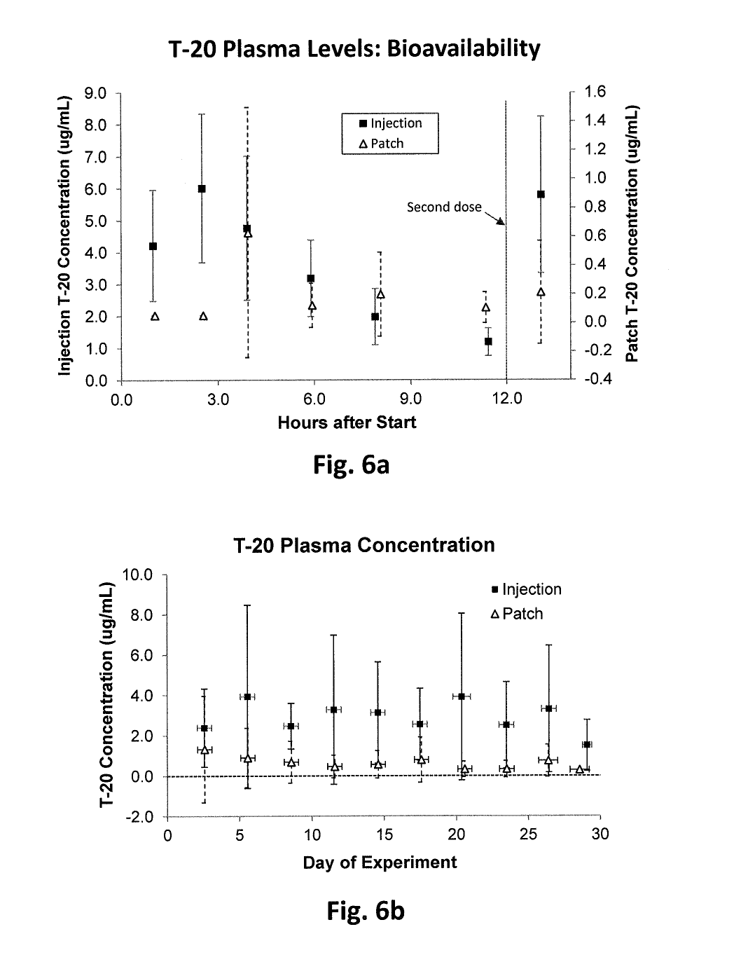

Results: The results are shown in FIGS. 6a-6b and FIGS. 7a-7b. The results demonstrate that ultrasound both successfully delivers T-20 through the skin and into the circulation, and does not significantly affect skin function. FIG. 6a presents exemplary data of blood plasma concentrations of T-20 delivered through animal skin in vivo over 30 days using a combination of the said embodiments of the passive and active patches and components. It specifically presents the concentration of T-20 in porcine plasma over a 12 hour period after the first 90 mg treatment and 1 hour after the second treatment for both transdermal delivery and subcutaneous injection. A time-dependent concentration can be seen in both cases, with a delayed peak for transdermal delivery indicating a more sustained release of T-20 into the circulation. FIG. 6b presents concentration of T-20 in porcine plasma as measured every three days before re-dosing. The concentration of T-20 delivered through the ultrasound-mediated match corresponds to approximately 30% of the bioavailability of the subcutaneous injection. Specifically, it shows that the concentration of T-20 in porcine plasma using transdermal delivery (0.6±0.2 ug/mL) was approximately 20-25% of the subcutaneous injection (2.8±0.8 ug/mL), which demonstrates that a significantly larger percentage of T-20, a large molecule, was delivered through the skin than the typical percentage of small molecules delivered passively in commercial transdermal patches.

FIG. 7a shows that the difference in TEWL from porcine skin at the treatment site and a control site prior to the first treatment with saline was not statistically different (N=6; p=0.53) and after 30-days was not statistically different (N=6; p=0.50). This demonstrates, relative to water loss function, that the skin was not affected by ultrasound alone (without T-20). FIG. 7b shows that the difference in TEWL from porcine skin at the treatment site and a control site prior to the first treatment with T-20 was not statistically different (N=13; p=0.76) and after 30-days was not statistically different (N=12; p=0.29).

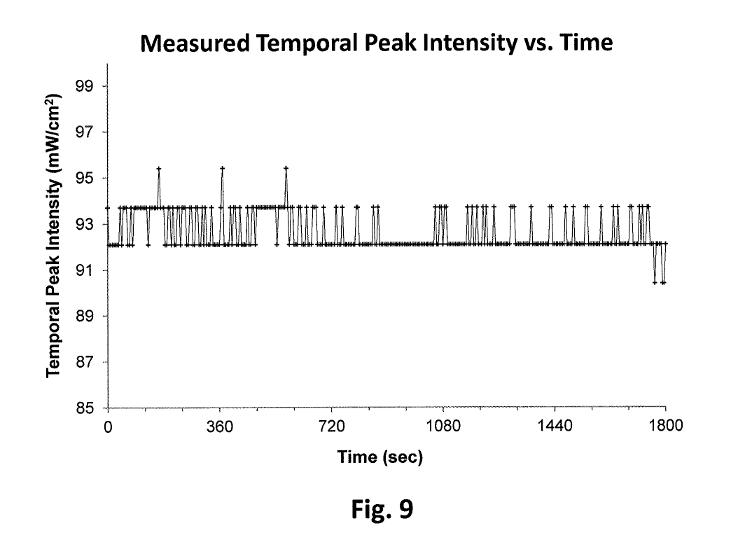

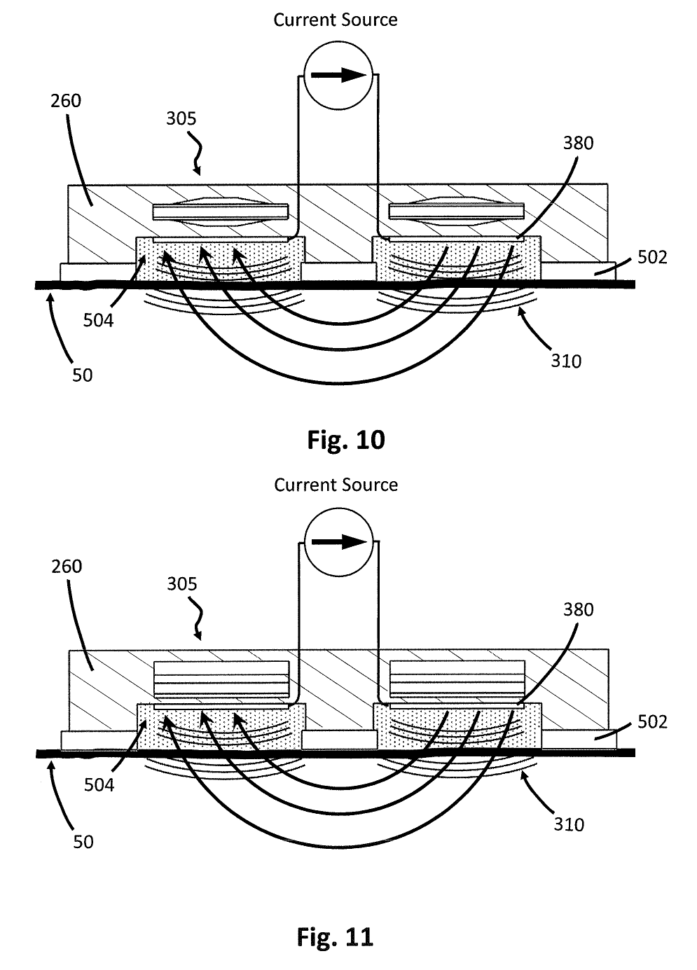

In at least one other embodiment, such as shown in FIGS. 10 and 11, ultrasound driven skin permeability is enhanced with iontophoresis in a single wearable patch to deliver at least one compound through the skin. Ultrasound changes skin properties to make it more permeable to large molecules which can be delivered by process of diffusion primarily. Iontophoresis uses an applied electric field that “pushes” charged molecules across the skin at rates higher than passive diffusion. For instance, the second active portion 300 may further include at least one electrode 380 in electrical communication with an electrical source (not shown) and positioned between the piezoelectric element(s) 200 and the substance(s) 504 to be delivered. In at least one embodiment, there are a pair of electrodes 380, each of a different electrical charge or polarity, such that when electrical current is applied to the electrodes 380, electrons flow from one electrode 380 to the other, as indicated in FIGS. 10 and 11. The electrode(s) 380 may include, but are not limited to, silver, chrome, gold, nickel, titanium, or any material capable of electrical conductivity, or combinations thereof.

In integrating the acoustic components, such as the piezoelectric elements 200 or flextensional transducers, with the iontophoresis components, such as the electrodes 380, care needs to be taken to ensure that the two sets of components do not interfere with each other. This could be accomplished by means such as, but not limited to, selecting the electrodes 380 to acoustically match the transducers or the potting material 260. The thickness, orientation and density of the electrodes 280 could be modified. The potting material 260 could also be made conductive, without altering its acoustic properties, by incorporation of a fraction of conductive material, or by choosing an inherently conductive polymer for the potting material 260. A polymer could also be used that becomes conductive when cross-linked or otherwise altered by exposure to a stimulus, such as, but not limited to, laser radiation. The same electrode 380 surface could also be used as part of a skin impedance monitoring electrode system that can control the delivery of ultrasound 310 to achieve the desired permeability for a variety of skin types/conditions. Algorithms can be developed to optimize the synergistic activity by applying only the amount of ultrasonic energy required to achieve sufficient permeability for the iontophoresis to work effectively on for the size of molecule involved. In this way, power consumption of the patch may be minimized while limiting the ultrasound exposure to ensure safe operation.

In another embodiment, the passive patch 500 may contain an embedded identifier, such as but not limited to a radio-frequency identification (RFID) tag, resistor or bar code, that identifies the patch as authentic, distinguishes the type of compound and/or distinguishes the dosage amount. When the active patch connects to the passive patch 500, the control box may identify the patch and use pre-programmed prescription information to ensure the proper patch, compound type and dosage are correct. The control box may adjust the duration and/or amplitude of treatment based on the information. The goal of this is to provide a “smart patch,” and eliminate the chance of accidental patch misuse if one patient is taking several transdermal drugs and to combat pharmaceutical fraud in which improper drugs are mis-labeled to trick consumers.

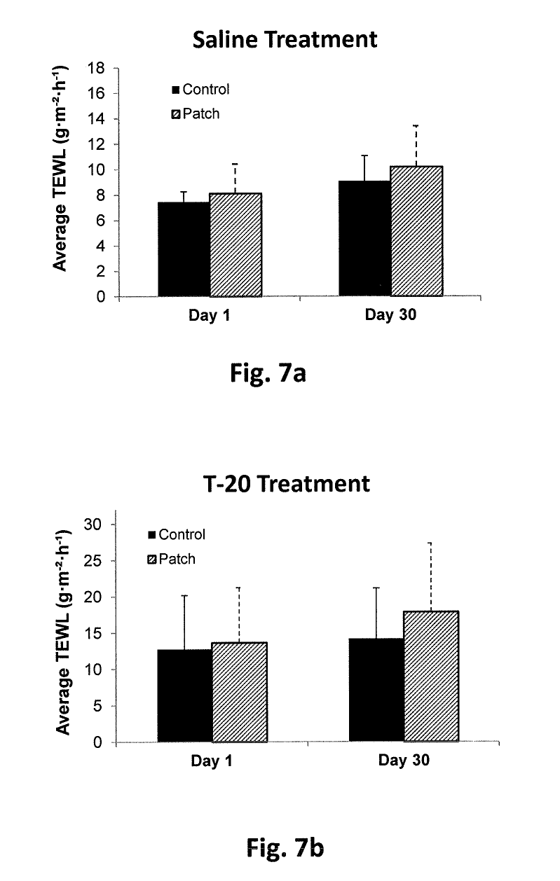

The control box, which in at least one embodiment is located externally of the active portion 300 or passive portion 500, may provide the electrical drive, voltage, current, or other electrical stimulation of the active patch component 300. It may further include electrical safety and matching circuitry, and capabilities of tracking of the active patch resonance characteristics. The system high level functions are controlled by the system control, as seen in FIG. 8a. This part of the system receives input from the user, such as enabling and disabling the drive voltage to the transducer/piezoelectric element. This can also include drive parameters, such as frequency limits and volt amplitude and phase angle that drive the transducer/piezoelectric element. This can also include the duty cycle and run time of the device. The system control also monitors running parameters and detects fault conditions, in which case the device output is disabled and the user is notified. The system control functions may be within a field programmable gate array (FPGA). The measurement circuitry measures the voltage and/or current of the transducer and translates it into phase angle measurements (theta, θ) and/or root-mean-square (RMS) values, which are then sent to the system control and phase controller. The phase angle controller compares the present phase angle to that set by the system control (θ_{ref}), which may be zero degrees in a preferred embodiment, to create an error signal. This error signal is sent to a control algorithm to produce the sinusoidal waveform at the frequency that minimizes the error signal frequency. This new waveform is then output to the transducer at the next drive cycle, and the voltage and current measurement is repeated. This control could specifically be an integration controller, but could also include a proportional integral derivative (PID) controller or other algorithm.

Signal generation is done using a switching amplifier that switches on and off quickly such as but not limited to a Class-D amplifier architecture. The input of the amplifier is a scaled sinusoid from a lookup table at the frequency set by the phase angle controller, as seen in FIG. 8b. This signal is filtered to remove the high frequency content inherent in the switching amplifier architecture to produce a clean sinusoid. The sinusoid is measured and driven into an impedance matching circuit attached to the transducer/piezoelectric element. The current drawn by a matching-transducer circuit is also measured at this point. Other waveforms could also be used to enhance skin permeabilization and/or compound delivery (via mechanical forces or acoustic streaming). The control box may provide two electrical connections from within one or more connectors, one of which may be electrical ground, though more preferentially both leads are floating relative to earth ground to provide electrical safety. The matching circuit, as seen in FIG. 8c, corrects the power factor and allows the amplifier to operate most efficiently at the designed impedance magnitude and frequency. The voltage into the matching circuitry is amplified to overcome any non-ideal (non-zero) phase angle of the transducer at the designed frequency. As a result of this configuration the switching amplifier can run at a low voltage (less than 30 volts) while the transducer operates at higher voltages, however if the transducer is removed or an electrical break occurs, the voltage will drop back down to safe, low voltages.

Changes in performance from aging of piezoelectrics, whether from long-term storage or long-term use, may also be accounted for by the control box system.

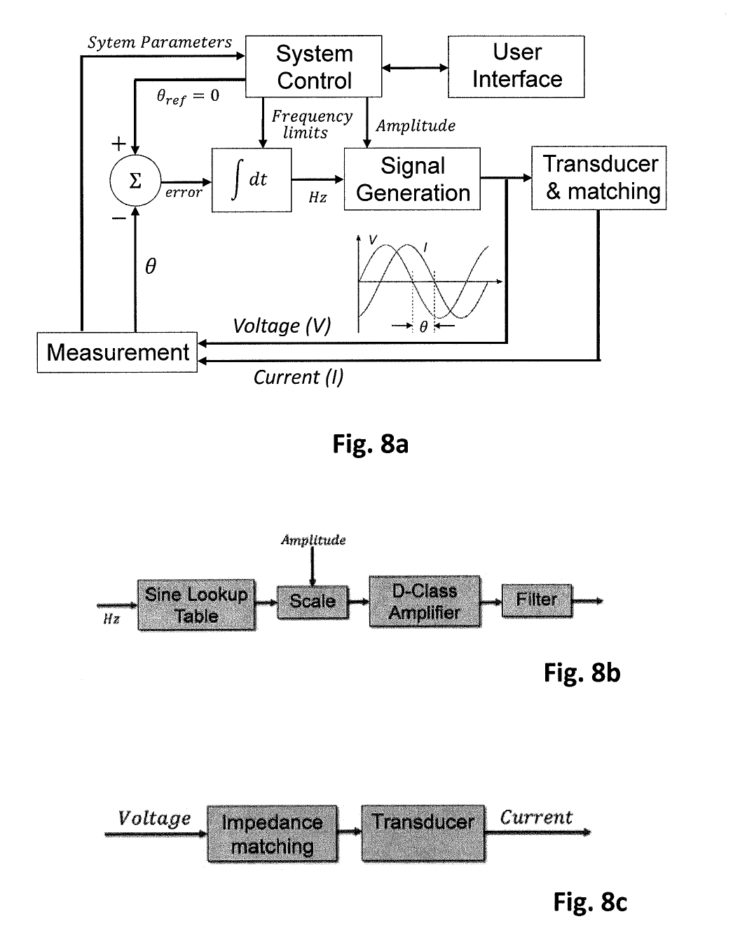

FIG. 9 shows the measured variation in spatial-peak temporal peak intensity of transdermal delivery system 100 of the present invention driven at 15% duty cycle by the control box over 30 minutes as measured by a calibrated hydrophone in a water tank. Measurement within a water tank using a calibrated hydrophone (Reson) demonstrated control of the output acoustic intensity to within ±2.5 mW/cm2 over 30 minutes by tracking the resonance frequency. The voltage amplitude of the sinusoidal signal output was kept fixed to a single value.

Another unique feature about the present invention is how the lightweight active patch 200 is combined with a passive patch 500 that contains the compound or substance 504 that will be transdermally delivered and the two can be separated so that the passive patch 500 delivers the compound for an extended period while the user is not tethered to the control electronics enabling user mobility. In one embodiment, the passive patch 500 will adhere to the skin with an adhesive 506 after a cover layer is removed, as shown in FIG. 1. Once adhered to the skin 50, a similar backing layer may be be removed upon which will expose an ultrasound gel vehicle or layer 508. The active patch 200 may then be placed on to the ultrasound gel layer 508 which increases the efficiency of transmission of the ultrasonic waves 310 from the piezoelectric element 200 where they are generated through the substance 504 and into the skin or tissue 50. Although described here as a gel, the ultrasound vehicle may be any material suitable for enhancing transmission of ultrasound waves. Beneath the ultrasound gel may be a film that is able to transmit the acoustic signal 310 through to the substance or compound 504, which is formulated in a way to permit the transmission of the acoustic signal through to the skin. In another embodiment, the ultrasound vehicle 508 may not be an integrated part of the first passive portion or patch 500, but rather may simply be applied to the first passive portion 500 (and/or to the second active portion 300) before attaching the first and second portions 500, 300 together.

How the active patch is fixed to the passive patch during treatment can include, but are not limited to, an elastic strap wrapping around the arm, leg, or torso, and a snap-in feature built into the passive and active patches. The active patch duration could be tailored to specific drugs or according to the users healthcare practitioner's recommendation, such as 30 minutes. Once the active patch duration is complete, it will be removed from the passive patch. The patch could also include a look up ‘dosing table’ where the user selects a compound, and the patch uses a pre-determined acoustic profile to permate the skin. The passive patch will then remain on the skin and continue to deliver the compound for as long as needed. Upon re-dosing, the current passive patch adhered to the skin would be removed and a new passive patch could be adhered to the same or different location. Once a new passive patch has been adhered, the active patch would then once again be used for the prescribed duration.

In another embodiment, an ultrasonic gel vehicle 508 is not needed. Rather, the passive patch 500 is adhered to the skin 50 after a cover layer is removed. Once adhered to the skin, a similar backing layer will be removed, upon which will expose the compound or substance 504. The active patch 300 would then be placed on to the passive patch 500 or compound 504 directly. The compound 504 would have to be formulated in a way to permit the transmission of the acoustic signal through to the skin.

In still yet other embodiments, the transducers/piezoelectric elements 200 could communicate at least two separate waves 310 into the living tissue 50 so as to interact along at least one region, thereby increasing the permeability of such tissues without irritation or damage thereto. In this sense, the separate waves 310 may be distinguished by different frequencies, intensities, durations, times of application, phases, or other characteristics of the ultrasound waves or application. The active patch 300 may also be set to deliver an initial dose of acoustic energy that removes or destroys any bacteria or undesired species on the skin that were not removed by alcohol wipes, or other methods.

In another embodiment, a hole, or series of holes, could be located through the thickness of the active patch 300, which could be used to pass through the potting material 260 (plastic or other) of the passive patch 500 and place a fiber optic or other sensor for monitoring skin or other conditions, such as temperature, oxygenation, or impedance.

In yet another embodiment, the individual cymbal transducers or other flextensional transducer 200 could have a backfill material inserted between the cap 201 and the piezoelectric element 200, to modify the acoustic properties or mechanical properties of the system, but without damping the motion of the cap to the point that the acoustic and displacement benefits are lost.

In another embodiment, the metal (or other material) caps 201 on the piezoelectric elements 200 could be modified with a selective laser cut or patterning that alters the acoustic profile delivered by the flextensional transducer 200. This effect could include, but is not limited to, increasing the flexibility or displacements of certain portions of the cap 201, or otherwise alters the density or properties of the acoustic field generated by the cap 201. Laser ablation or machining could also be used to tune a cap on the ‘front’ of a transducer to better match (or mismatch) the cap on the ‘back’ of the transducer.

In another embodiment, the potting material 260 could be machined with a series of grooves or holes that alter the acoustic and mechanical properties, including a better acoustic match to the target (skin or other). A mechanical device, such as a clamp or metal band, could be used to selectively compress or pull on the potting material 260, to adjust mechanical and acoustic properties.

The active patch 300 and potting material 260 could also be manufactured from a material that can be reversibly modified, using light or electrical stimulation, for example, to temporarily change its acoustic properties.

A second acoustic field, at another intensity or wavelength, could be applied by the system after treatment is complete, to close the pores, if desired.

The active patch 300 could be tuned to the response of the skin of an individual patient. This process could include, but is not limited to a) having a clinician apply the patch with compound to the skin b) the clinician applies a level of acoustic energy c) the clinician measures the content of the compound in the tissue, blood, or other diagnostic, and d) the clinician continues to increase the acoustic energy to reach the minimum intensity to open the skin, and sets the patch for the patient at this level.

Approaches for measuring dosing, if needed, could include an infrared signal through the compound to monitor liquid levels or active (compound) species, a signal (such as a strain gauge) that measures the volume inside the patch, or by measuring conductivity inside the passive compound pass which would change as ionic species leave the passive patch and enter the skin.

The invention could also incorporate a second ‘scout’ patch that monitors the skin properties separate from the area under the acoustic patch and provides feedback to the acoustic patch and control system (e.g., the patient is hot, sweating, dehydrated) that could affect the dosing profile. Several advantages are offered by the invention. The invention facilitates the needle-free, automated and safe delivery of nutrients and other fluids required to treat open wounds. The invention minimizes the risk of infection otherwise caused by needles. The invention can be integrally manufactured, including lightweight and compact power electronics and control mechanisms, so as to have a small footprint to minimize the tissue area affected by the device and to minimize discomfort to the wearer, thus providing a compact, wearable solution. The invention offers a wide range of power solutions, including propane or hydrogen fuel cells, batteries, and DC power via a wall outlet. The invention is readily adaptable to a variety of computers via an interface to monitor and control the reservoir, flow from the reservoir, and flow into the user.

Since many modifications, variations and changes in detail can be made to the described embodiments presented herein, and which are still within the scope of the invention, it is intended that the present descriptions in the accompanying drawingsand specification, be interpreted as illustrative and not in a limiting sense. Thus, the scope of the invention should be determined by the appended claims and their legal equivalents. Now that the invention has been described,

What is claimed is:

1. An ultrasonic source comprising:
at least one piezoelectric element having a first surface and an opposite second surface;
a support member having a first side receiving and restraining said second surface of said at least one piezoelectric element and an opposite second side; and
at least one fulcrum secured to said second side of said support member and restricting movement of at least a portion of said at least one piezoelectric element when said at least one piezoelectric element is activated.

2. The ultrasonic source as recited in claim 1, further comprising a cymbal cap affixed to said first surface of said at least one piezoelectric element.

3. The ultrasonic source as recited in claim 1, wherein said first surface of said at least one piezoelectric element moves radially relative to said second surface when said at least one piezoelectric element is activated.

4. The ultrasonic source as recited in claim 3, wherein at least a portion of said at least one piezoelectric element moves longitudinally relative to said at least one fulcrum when said at least one piezoelectric element is activated.

5. The ultrasonic source as recited in claim 4, wherein said at least one portion of said at least one piezoelectric element is distal from said fulcrum.

6. The ultrasonic source as recited in claim 1, further comprising a plurality of piezoelectric elements each having a first portion of positive polarity and a second portion of negative polarity, wherein said plurality of piezoelectric elements are positioned adjacent to one another in alternating polarity.

7. The ultrasonic source as recited in claim 6, wherein said plurality of piezoelectric elements are electrically connected in parallel.

8. The ultrasonic source as recited in claim 6, wherein said plurality of piezoelectric elements are electrically connected in series.

9. A transdermal delivery system, comprising:
a first portion including at least one reservoir containing at least one substance to be delivered to a tissue, said first portion contacting the tissue; and
a second portion positioned adjacent to said first portion opposite of the tissue, said second portion including at least one ultrasonic source in transmitting communication of at least one ultrasound vibration through said at least one reservoir and into the tissue, said at least one ultrasonic source comprising
(i) at least one piezoelectric element having a first surface and an opposite second surface;
(ii) a support member having a first side receiving and restraining said second surface of said at least one piezoelectric element and an opposite second side; and
(iii) at least one fulcrum secured to said second side of said support member and restricting movement of at least a portion of said at least one piezoelectric element when said at least one piezoelectric element is activated.

10. The transdermal delivery system as recited in claim 9, wherein said second portion is selectively releasable from said first portion.

11. The transdermal delivery system as recited in claim 9, wherein said first portion is disposable and said second portion is reusable.

12. The transdermal delivery system as recited in claim 9, wherein said first portion further comprises at least one membrane disposable in contacting between the tissue and said reservoir, said at least one membrane permeable to said at least one substance upon application of said ultrasound vibration for movement of said at least one substance from said reservoir into the tissue.

13. The transdermal delivery system as recited in claim 9, further comprising an ultrasonic coupling vehicle between said first portion and said second portion.

14. The transdermal delivery system as recited in claim 9, further comprising a control unit in electrical communication with said second portion, detecting an impedence of said second portion, providing electrical stimulation to said at least one piezoelectric element, detecting a difference between electrical stimulation supplied to said second portion and current returning from said second portion, and adjusting said electrical stimulation supplied to said at least one piezoelectric element for activation to compensate for said difference.

15. The transdermal delivery system as recited in claim 9, wherein said second portion further comprises at least one electrode in electrical communication with an electrical source and positioned between said at least one piezoelectric element of said second portion and said at least one substance.
